(19) United States
(12) Patent Application Publication
(10) Pub. No.: US 2003/0109512 A1
(43) Pub. Date: Jun. 12, 2003

(54) Use of 4-androstenediol and 4-androstenediol in a transdermal formulation to increase testosterone levels in humans

(76) Inventors: Andrew Kucharchuk, Baton Rouge, LA (US); Merrill A. Patin, Baton Rouge, LA (US)

Correspondence Address: THOMAS S. KEATY, KEATY PROFESSIONAL LAW CORP., 2140 WORLD TRADE CENTER, NO. 2 CANAL STREET, NEW ORLEANS, LA, 70130, US

(21) Appl. No.: 10/281,539

(22) Filed: Oct. 29, 2002

Related U.S. Application Data
(62) Division of application No. 10/021,114, filed on Dec. 12, 2001, now abandoned.

(51) Int. Cl. A61K 31/57; A61K 31/405; A61K 31/315

(52) U.S. Cl. 514/178; 514/419; 424/449; 514/494

(57) ABSTRACT

A method of increasing testosterone level in humans by topical application of a cream or gel involves the use of an androgenic testosterone precursor and a protein precursor for elevating the symptoms of depression and lethargy associated with low level testosterone. The androgenic precursor is 4-androstenediol, the protein precursor is 5-hydroxytryptophan. The two active ingredients are mixed in therapeutically effective amounts with a penetrating cream or gel containing a formulation of lecithin, isopropyl palmitate, and pluronic.

The resultant composition of matter is applied as a transdermal formulation. The testosterone level is increased, while the toxicity problems associated with conventional administration of testosterone are avoided.

### BACKGROUND OF INVENTION

The present invention relates to a method of administering the testosterone precursor 4-androstenediol for increasing testosterone level in humans. Human males naturally produce the hormone testosterone substantially during the lifetime of the person. As the body ages, the level of testosterone slowly declines, causing bone mass loss, sexual dysfunction, lethargy, loss of muscle mass, and strength.

To slow down the undesirable effects of aging, various techniques have been developed. Some of the techniques involve the process of introducing prohormones that are designed to increase the testosterone level. This process includes the use of oral preparations, wherein a therapeutic agent is administered in the form of a pill. The major drawback of oral preparations of testosterone is the fact that they must be in a form that will allow their absorption from the gastrointestinal tract. The prohormones administered by the oral method are taken in an inactive form, absorbed and then activated in the liver in a process known as methylation. As can be expected, these preparations are fraught with liver dysfunction.

The testosterone elevating drugs can be also introduced by injections. This is by far the most popular method of testosterone supplementation. The testosterone injections had been used since World War II. These provide non-physiological blood levels; either supra physiological or infra-plhysiological, depending on the temporal proximity to the injection. Most often used compounds are testosterone cypionate and testosterone enanthate. These compounds are administered as a 200-mg injection every two to three weeks. The drawback of the injection method is that an initial super-physiologic testosterone level the first few days after the injection is followed by a decrease. The resultant peak-and-valley effect may have more long-term consequences than a steady level that may be produced by transdermal preparations.

Other types of testosterone preparations include testosterone pellets that can be implanted under the skin of the patient. Still other methods involve the use of sublingual preparations where the patient places the medicine under the tongue, as well as testosterone patch.

One of the known methods of increasing testosterone method in humans relates to the use of an androgenic steroid androstenediol. Some studies suggest that androstenediol may increase endogenous testosterone levels by 150% in 15 minutes if administered properly. Androstenediol is considered a nutriceutal or nutritional product since it is found in meats and plants, such Mexican wild yams. Under the interpretation of the Federal Drug Administration of the Dietary Supplement Act, androstenediol is considered a dietary supplement as long as it was not previously marketed as a drug.

One of the methods of administering androstenediol method is disclosed in U.S. Pat. No. 5,880,117 issued on Mar. 9, 1999 to Arnold. According to the '117 patent, 4-androstenediol is administered orally in daily doses of 25 milligrams to 2500 milligrams. The 4-androstenediol compound in the '117 patent is 4-androstene-3beta, 17betadiol.

Another method of increasing testosterone levels in humans is disclosed in U.S. patent application Ser. No. 2001-0008638, published on Jul. 19, 2001, inventor Wilding. According to the application method, an oral dosage delivery system is used for introducing a therapeutic agent in the form of an androgen derivative. The compound includes prohormnone/anti-estrogen mixture in a time-released or controlled released dosage form. According to the application, the ingested compound is absorbed into the human digestive system to slowly penetrate the blood stream over a 6-8 hour period. At least one testosterone prohormone is selected from the group consisting of 19 Nor-4-androstene-3, 17 dione; 4-androstene-3.beta, 17.beta diol; 5-androstene-3.beta, 17.beta.diol; and Tribulus terrestris. The androgen prohormnones are combined with at least one anti-estrogen ingredient selected from the group consisting of chrysin, indole-3-carbinolel, saw palmetto extract, and mixtures thereof. According to the application, the oral dosage is sufficient to sustain elevated blood serum testosterone levels for a period over 6 hours.

Still another method of increasing the testosterone level in males involves the use of an oral synthetic testosterone (methyltestosterone). This hormone is believed to increase the risk of toxicity because of its structural modifications when introduced into the blood stream.

In clinical trials it was determined that peroral androstenediol was superior as an androgen to androstenedione; however, it still has the disadvantages of an oral supplement. As with many other oral medications, the blood level varies significantly; the precursors are slow to metabolize, which is considered a distinct disadvantage for persons taking the medication before a strenuous exercise or a sexual activity. An additional disadvantage presented by oral medication before a competitive event is the compliance issue with anti-drugs policy adopted by most sport authorities.

The present invention contemplates elimination of drawbacks associated with the prior art and provision of a therapeutic agent suitable for use in increasing testosterone levels in humans, while providing a more reliable therapeutic response.

### SUMMARY OF THE INVENTION

It is, therefore, an object of the present invention to provide a composition of matter suitable for use as a therapeutic agent for increasing testosterone levels in humans.

It is another object of the present invention to provide a therapeutic agent that can get administered topically.

It is still a further object of the present invention to provide a non-toxic natural therapeutic agent that can be quickly metabolized in the system of the user.

It is still a further object of the present invention to provide a therapeutic agent that can raise testosterone levels higher and faster than conventional oral administration of androstenedione or androstenediol.

It is still another object of the present invention to provide a compound that can be mixed with a mood-elevating agent to further decrease the symptoms of depression and lethargy.

These and other objects of the present invention are achieved through a provision of a composition of matter comprising therapeutically effective amounts of an androgenic testosterone precursor mixed with a protein precursor in a cream base. The mixture is administered topically by rubbing into the skin of the user.

The androgenic testosterone precursor is preferably 4-androstenediol and the protein precursor is 5-hydroxytryptophan. In the most preferred embodiment, the 4-androstenediol is 4-androstene-3beta, 17 betadiol. The mixture of the two active ingredients is combined with a penetrating cream that comprises pluronic lecithin organo (PLO) cream to facilitate percutaneous penetration of the therapeutic substances into the user's blood stream.

The formulation of the present invention comprises between about 5% to about 30% by weight of 4-androstenediol, between about 0.5% to about 10% by weight of zinc gluconate USP and between about 0.5% to 2.5% by weight of hydroxytryptophan (L-5), mixed with 10% by weight of ethoxy diglycol reagent. The resultant mixture is then mixed with between about 20% to about 22% by weight of lecithin isopropyl palmitate solution dissolved in 20% solution of Pleuronic F127 to make up 100% of the formulation.

The cream is applied 30-60 minutes before an anticipated strenuous or sexual activity, this time being sufficient for the active ingredients to penetrate through the skin of the patient. The formulation and the delivery method of the present invention allow for the stable and reliable increase in testosterone level to natural levels, while relieving the symptoms of lethargy and depression associated with low level testosterone.

The transdermal introduction of testosterone enhancing formulation has a lower incidence of side effects as compared to oral preparations, which may affect liver function of the user. With the transdermal application the testosterone levels are predictable, unlike the unpredictable peak and valley modulations associated with the injection methods. The androgenic steroid may be administered in a lower dose for achieving the beneficial therapeutic effect.

Males seeking a non-evasive therapy for the symptoms of lower level testosterone and associated symptoms of depression or lethargy can use the formulation of the present invention. The transdermal application avoids passing of the hormone through the liver because 3beta-hydroxysteroid dihydrogenase enzyme converts the 4-androstenediol into testosterone before it has a chance of reaching the liver. As a result, various toxicity problems are successfully avoided.

### DETAILED DESCRIPTION OF THE PREFERRED EMBODIMENT

In accordance with the present invention, an effective amount of an androgenic steroid androstenediol, more particularly 4-androstenediol is introduced in a transdermal formulation. 4-androstenediol is a naturally occurring compound, a metabolite of testosterone in placenta, testicular adrenal and hypothalamic-pituitary tissues. In a human body 4-androstenediol converts to testosterone through the 3beta-hydroxysteroid dihydrogenase enzyme.

Generally, 4-androstenediol refers to two isomers, 4-androstene-3beta, 17 betadiol and 4-androstene-3alpha, 17beta-diol. The present invention, in its preferred embodiment utilizes 4-androstene-3beta, 17betadiol. In its natural form, it is a crystalline androgenic steroid.

The transdermal compound of the present invention includes the androgenic testosterone precursor 4-androstenediol combined with a mood elevating substance, 5-hydroxytryptophan (L-5) and mixed with a transdermal carrier to form the homogenous cream.

The mood elevating substance selected for use in the present invention contains an amino acid tryptophan. Several studies suggest that the protein precursor tryptophan provides relief from depression and acts as a mood elevating substance. Tryptophan is found in natural products and dietary supplements. Tryptophan-containing foods are reported to boost the level of the amino acid involved in the production of serotonin. Serotonin is a neurotransmitter important for sleep, positive moods, and for a number of other conditions essential for well being. Serotonin is a natural chemical produced by the human brain. Hydroxytryptophan L-5 (5-hydroxytryptophan) was reported as beneficial for insomnia, headaches, and depression. While high does of serotonin taken orally may have adverse effects on the hemosystem, transdermal administration of the serotonin-producing substance is believed to be safer and more reliable.

According to the present invention, effective amounts of 4-androstenediol and 5-hydroxytryptophan are mixed with a transdermal cream that facilitates delivery of the active agents into the blood stream of the user. It is believed that the androgenic steroid converts to testosterone via the 3-beta hydroxysteroid dehydrogenase enzyme without passing through the liver.

In the preferred embodiment of the present invention, lecithin isopropyl palmitate solution was used. Soy lecithin/isopropyl palmitate is used in topical pharmaceutical and cosmetic preparations as a non-oleaginous emollient with good spreading characteristics. It is a clear, odorless viscous liquid that solidifies at less than 16 degrees Celsius. Lecithin is a complex mixture of phospholipids and other materials. The mixture may be produced in a physical form from viscous to semi-liquid to powder, depending upon the free fatty acid content.

Generally, lecithins are widely used as dispersing, emulsifying and stabilizing agents. The combination of lecithin, isopropyl palmitate, and pluronic F-127 is useful in pharmaceutical compounds due to the solubilizing and penetration enhancement qualities. As used herein, the term “penetration enhancement” means that the materials have a direct effect on the permeability of the skin's barrier. The transdermal cream of the present invention contains Lecithin, Isopropyl palmitate, and pluronic F-127 solution designed to increase percutaneous absorption of the active ingredients by the skin and ultimately into the blood stream of the user. It is believed that a pluronic-lecithin-organo compound (PLO cream or gel) helps prepare the skin and allow for effective transport of the active ingredients into the user's bloodstream.

The formulation of the present invention comprises between about 5% to about 30% by weight of 4-androstenediol, between about 0.5% to about 10% by weight of zinc gluconate USP and between about 0.5% to 2.5% by weight of hydroxytryptophan (L-5), mixed with 10% by weight of ethoxy diglycol reagent. The resultant mixture is then mixed with between about 20% to about 22% by weight of lecithin isopropyl palmitate solution dissolved in 20% solution of pluronic F127 to make up 100% of the formulation.

To prepare the preferred embodiment of the transdermal cream used in the present invention, 100 grams of pluronic F127 20% solution were mixed with 1.5 grams of potassium sorbate NF, a preservative. Pluronic F-127 NF and potassium sorbate NF were thoroughly mixed with refrigerated distilled water to make up 500 ml. The mixture of powders and distilled water was then placed under refrigeration for a period of 24 hours to allow for complete dissolution of ingredients.

Lecithin isopropyl palmitate solution was prepared by mixing 250 grams of lecithin soya in a granular form with 292 ml of isopropyl palmitate NF, and 1.32 gms of sorbic acid NF-FCC powder. The three ingredients were thoroughly mixed in a large beaker, covered with aluminum foil and allowed to sit for 24 hours until full dissolution of the ingredients was achieved.

Next, the carrier cream was mixed with the active ingredients. In this step of the process, for each 100 gms of the finished product, 15 gms of 4-androstenediol, 5 gms of zinc gluconate USP and 1 gm of hydroxytryptophan (L-5) were mixed and reduced to fine particle size by triturating with appropriate equipment. 10 ml of ethoxy diglycol reagent were levigated with dry ingredients to form a uniform mixture. It is believed that the trace metal zinc helps in the anti-estrogen effect of the transdermal formulation of the present invention.

Then, 21.7 mls of lecithin isopropyl palmitate solution were thoroughly mixed with the 4-androstenediol mixture to form a uniform compound. Finally, the resultant mixture of 4-androstenediol with the lecithin isopropyl palmitate solution was added, with continuous mixing to pleuronic F-127 20% solution to make up 100 ml of the final product. The mixture forms a transdermal cream or gel of the present invention.

Clinical trials were conducted by administering the topical androstenediol in a PLO cream base to 17 middle-aged males before exercise or sexual encounter. The daily doses can range from 25 milligrams to 500 milligrams with 50 milligrams to 100 milligrams daily dosage being preferable. Th cream was applied to the inside wrist skin area of the user.

The subjects experienced maximum effect in 60 minutes. Generally, the effects of the supplement lasted 3-4 hours before returning to a baseline. It is believed that topical formulation of the present invention may be given 30 minutes before exercise or sexual activity.

The recommended daily doses of the 4-androstenediol may range from 50 mg to 250 mg. The 5-hydroxytryptophan may be administered in a daily dose of up to 750 mg.

The transdermal administration of the testosterone elevating formulation avoids the toxicity factor associated with oral administration of testosterone or testosterone injections. With the transdermal introduction of testosterone elevating agents, it is believed that there is no need to monitor prostate-specific antigen levels.

It is also believed that transdermal introduction of testosterone enhancing formulation has a lower incidence of side effects as compared to oral preparations, which may affect liver function of the user. It is also believed that with the transdermal application the testosterone levels remain stable, unlike the unpredictable peak-and valley modulations associated with the injection methods.

The transdermal formulation of the present invention requires a lower dose of steroid; the user without a specific medical training may apply it by simply rubbing the required dose of the cream to the skin of the user. The PLO cream carrier helps in the process of transporting the active ingredients into the blood of the user.

The mood-enhancing agent further relives the symptoms of lethargy and depression. When administered in a transdermal formulation, it is believed to have a more stable release time. It is also believed that the use of 4-androstenediol in a transdermal formulation is superior to various topical preparations containing androstenedione. Some studies indicate that androstenedione also causes elevation of estrogen levels, which may increase formation of fatty tissue in the body without increasing the muscle tissue.

The product and method of application of the present invention offers to users an option of increasing blood flow testosterone levels in a controlled manner, without exceeding the natural levels that could be prohibited by sporting authorities conducting tests at competition events. The formulation of the present invention can be also be used by males seeking a non-evasive therapy for the symptoms of lower level testosterone and associated depression or lethargy. The transdermal application avoids passing of the hormone through the liver because 3beta-hydroxysteroid dihydrogenase enzyme converts the 4-androstenediol into testosterone before it has a chance of reaching the liver. As a result, various toxicity problems are successfully avoided.

It is anticipated that aging males who are showing signs of de-conditioning, decreased libido and loss of energy will particularly benefit from the formulation and the application method of the present invention. Other potential beneficiaries are athletes, especially sportsmen engaged in a prolonged competition event, such as triathlon, marathon, etc.

The composition of matter of the present invention may have therapeutic effect on people interested in an anabolic effect since it requires another enzyme (17BHSD) for conversion. As such, sportsmen desiring to increase lean muscle mass without increasing the estrogen level may advantageously use the product.

The formulation of the present invention may be contraindicated for women and men with hormone dependent cancers, such as breast or prostate cancers, or for men having a predisposition to prostate cancer.

Many changes and modifications can be made in the formulation and method of the present invention without departing from the spirit thereof. We, therefore, pray that my rights to the present invention be limited only by the scope of the appended claims.

We claim:

1. A method of increasing testosterone level in humans by transdermal administration of 4-androstenediol.

2. The method of claim 1, wherein the 4-androstenediol is 4-androstene-3beta, 17betadiol.

3. The method of claim 1, wherein 4-androstenediol is mixed with a mood-elevating composition for relieving the symptoms of depression and lethargy associated with low level testosterone.

4. The method of claim 3, wherein a mixture of 4-androstenediol and the mood-elevating composition is combined with a penetrating cream to facilitate absorption of the mixture into a human body.

5. The method of claim 3, wherein said mood-elevating composition is 5-hydroxytryptophan.

6. The method of claim 1, wherein said 4-androstenediol is administered in a transdermal daily dosage of 50 mg to 250 mg.

7. The method of claim 5, wherein the 5-hydroxytryptophan is administered in a daily dosage of up to 750 mg.

8. The method of claim 4, wherein said penetrating cream is pluronic lecithin organo cream.

9. The method of claim 5, wherein said mixture comprises between about 5% to about 30% by weight of 4-androstenediol and between about 0.5% to 2.5% by weight of 5-hydroxytryptophan.

10. The method of claim 9, wherein said mixture further comprises between about 0.5% to about 10% by weight of zinc gluconate and about 10% by weight of ethoxy diglycol reagent, mixed with a penetrating cream.

11. The method of claim 10, wherein said penetrating cream comprises lecithin, isopropyl palmitate, and pluronic.

12. A method of increasing testosterone level in humans and treating the symptoms of lethargy associated with low level testosterone comprising the steps of administering by a transdermal application an effective amount of mixture of 4-androstenediol and 5-hydroxytryptophan, mixed with a penetrating cream.

13. The method of claim 12, wherein said 4-androstenediol is administered in a transdermal daily dosage of 50 mg to 250 mg.

14. The method of claim 12, wherein the 5-hydroxytryptophan is administered in a daily dosage of up to 750 mg.

15. The method of claim 12, wherein said penetrating cream comprises pleuronic lecithin, isopropyl palmitate, and pluronic.

16. The method of claim 12, wherein said mixture comprises between about 5% to about 30% by weight of 4-androstenediol and between about 0.5% to 2.5% by weight of 5-hydroxytryptophan.

17. The method of claim 16, wherein said mixture further comprises between about 0.5% to about 10% by weight of zinc gluconate and about 10% by weight of ethoxy diglycol reagent, mixed with lecithin, isopropyl palmitate, and pluronic.

18. A composition of matter for increasing testosterone level in humans and decreasing the symptoms of depression and lethargy associated with low testosterone level, said composition of matter comprising therapeutically effective amounts of an androgenic testosterone precursor and a protein precursor.

19. The composition of matter of claim 18, further comprising a pharmaceutically suitable carrier.

20. The composition of matter of claim 19, wherein said pharmaceutically suitable carrier is a dermatologically suitable carrier.

21. The composition of matter of claim 20, wherein said dermatologically suitable carrier comprises pluronic lecithin organo cream.

22. The composition of matter of claim 18, wherein said androgenic testosterone precursor is 4-androstenediol.

23. The composition of matter of claim 18, wherein said protein precursor is 5-hydroxytryptophan.

24. The composition of matter of claim 18, wherein said mixture of the androgenic testosterone precursor and the protein precursor comprises between about 5% to about 30% by weight of 4-androstenediol and between about 0.5% to 2.5% by weight of 5-hydroxytryptophan.
